(19) United States
(12) Patent Application Publication
(10) Pub. No.: US 2024/0156842 A1
(43) Pub. Date: May 16, 2024

(54) Topical NSAID Formulation with Improved Skin Absorption

(71) Applicant: William Spivak, Englewood, NJ (US)

(72) Inventor: William Spivak, Englewood, NJ (US)

(21) Appl. No.: 18/505,309

(22) Filed: Nov. 9, 2023

Related U.S. Application Data
(60) Provisional application No. 63/383,853, filed on Nov. 15, 2022.

(51) Int. Cl. A61K 31/618; A61K 9/00; A61K 31/045; A61K 31/125; A61K 31/192; A61K 31/352; A61K 36/28; A61K 47/20; A61P 19/02; A61P 29/00

(52) U.S. Cl. CPC A61K 31/618; A61K 9/0014; A61K 31/045; A61K 31/125; A61K 31/192; A61K 31/352; A61K 36/28; A61K 47/20; A61P 19/02; A61P 29/00

(57) ABSTRACT

A topical therapeutic composition comprising naproxen and menthol. The naproxen penetrates through the skin for local effect against pain and inflammation (e.g. at a joint) as well as systemic absorption. The menthol could work as both an analgesic and to enhance skin penetration of the naproxen. The composition could further comprise other therapeutic ingredients. For example, the composition could further comprise a different analgesic such as camphor or methyl salicylate. In another example, the composition could further comprise a botanical ingredient such as Arnica Montana or Burdock root. In another example, the composition could further comprise a different skin penetration enhancing agent such as DMSO. The topical therapeutic composition could be used for the treatment of various conditions such as neuromuscular, skeletal, or arthritic disorders, or for the treatment or prevention of Alzheimer disease.

### TECHNICAL FIELD

This invention relates to a topical drug formulation of naproxen.

### BACKGROUND

Pain from musculoskeletal problems such as osteoarthritis of joints, muscle strain, or tendon strain, will affect almost every person during their lifespan. As an example, low back pain affects more than 25% of the adult population in the U.S. at any given time. Low back pain has a huge financial impact on lost wages and medical care. Chronic low back or joint pain are often treated by expensive procedures such as steroid injections into the affected areas (possibly under fluoroscopy). Such injections may temporarily relieve pain but can lead to further bone degeneration and involve exposure to ionizing radiation. More severe cases of chronic musculoskeletal pain may be treated with expensive surgical procedures such as laminectomy or spinal fusion, or joint replacement (e.g. knee or hip). However, these often only temporarily improve the mechanics of the spine or joint, and do nothing to arrest the progressive nature of joint degeneration. Progression of osteoarthritis can further lead to joint destruction, nerve compression, and further debilitating symptoms. Despite being widespread, musculoskeletal problems have no optimal treatment. Less invasive and inexpensive treatment options are needed. Also see bibliographic references listed in the priority provisional patent application Ser. No. 63/383,853 (filed 15 Nov. 2022).

### SUMMARY

NSAID. In one aspect, this invention is a topical therapeutic composition comprising a non-steroidal anti-inflammatory drug (NSAID, see below for more details). The therapeutic composition comprises the NSAID in a therapeutically-effective amount. In a preferred embodiment, the NSAID is naproxen. The amount of naproxen could constitute 1-25 wt % or 3-18 wt % or 3-12 wt % of the total weight of the composition. As used herein, the term “naproxen” encompasses both the free acid form or a pharmaceutical salt form thereof such as naproxen sodium. Naproxen sodium may be preferred over naproxen free acid because of better skin penetration.

The amounts for the ingredients of the therapeutic composition could alternately be expressed as weight/volume concentration (mg/ml). Selection of measurement units is a matter of convenience or preference. For example, lower viscosity liquid compositions such as sprays are more easily measured by volume. However, higher viscosity compositions such as creams and lotions are more easily measured by weight. These measurements are interconvertible by empirical testing, density modeling, or other physiochemical techniques. The concentration of naproxen in the therapeutic composition could be 10-250 mg/ml or 30-180 mg/ml or 30-120 mg/ml.

Topical Analgesic. In addition to the NSAID, the therapeutic composition could further comprise one or more topical analgesics in a therapeutically-effective amount. One example of a topical analgesic that could be used is menthol (see below for more details) provided in a therapeutically-effective amount. Note that menthol could also be considered as a penetration enhancing agent (see below for more details). The amount of menthol could constitute 1-25 wt % or 3-18 wt % of the total weight of the composition. Alternately, the amount of menthol could be as weight/volume concentration. The concentration of menthol in the therapeutic composition could be 10-250 mg/ml or 30-180 mg/ml.

Another example of a topical analgesic that could be used is camphor (see below for more details) provided in a therapeutically-effective amount. The amount of camphor could be 2-15 wt % or 2-10 wt % of the total weight of the composition. Alternately, the amount of camphor could be expressed as weight/volume concentration. The concentration of camphor in the therapeutic composition could be 20-150 mg/ml or 20-100 mg/ml.

Another example of a topical analgesic that could be used is a salicylate (see below for more details) provided in a therapeutically-effective amount. In a preferred embodiment, the salicylate is methyl salicylate. The amount of methyl salicylate could constitute 10-50 wt % or 15-40 wt % of the total weight of the composition. Alternately, the amount of methyl salicylate could be expressed in weight/volume concentration. The concentration of methyl salicylate in the therapeutic composition could be 100-500 mg/ml or 150-450 mg/ml. Other examples of topical analgesics that could be used include aloe vera, wintergreen, acetylsalicylic acid, and capsaicin.

Botanical Ingredient. In addition to the NSAID, the therapeutic composition could further comprise one or more botanical medicinal ingredients in a therapeutically-effective amount. The botanical medicinal ingredient may be added to the composition in any suitable form such as liquid extract (e.g. aqueous or alcohol), resin, dried powdered, or oil. The botanical medicinal ingredient could be from any part of the plant such as flower, stem, leaf, root, etc.

One example of a botanical ingredient that could be used is Arnica Montana (see below for more details). The amount of Arnica Montana could constitute 1-15 wt % of the total weight of the composition. Another example of a botanical ingredient that could be used is Burdock root (Arctium Lappa, see below for more details). The amount of Burdock root could constitute 1-15 wt % of the total weight of the composition.

Other examples of botanical ingredients that could be used include Aloe Barbadensis (vera), Boswellia Carterii, Calendula Officinalis, Camellia Sinensis (green tea), Ilex Paraguariensis, Melissa Officinalis (lemon balm), Boswellia Serrata, Eucalyptus Globulus, peppermint, and olive fruit.

Other Active Pharmaceutical Ingredient (API). The composition may further include one or more of any other APIs that can be used topically, such as those which are anti-inflammatory agents. Examples of anti-inflammatory agents that could be used in the composition include cromolyn or quercetin (see below for more details).

Other Ingredients. The topical therapeutic composition could have other ingredients for pharmaceutical purposes. Examples include dimethicone; ethylenediaminetetraacetic acid (EDTA, which is a chelating agent that acts as a preservative by binding metal ions that could otherwise cause metal-catalyzed degradation); cyclomethicone; glycine; silica; chlorphenesin (preservative); pH adjusting agents (for acidifying, alkalizing, or buffering) to maintain a desired pH; and vitamin E, tocopherol, and its various derivatives, such as tocopheryl acetate, tocopheryl linoleate, tocopheryl linoleate/oleate, tocopheryl nicotinate, tocopheryl succinate, and potassium ascorbyl tocopheryl phosphate.

Penetration Enhancing Agent. The therapeutic composition could further comprise one or more skin penetration enhancing agents that act to improve absorption through the skin. There are a variety of different types of penetration enhancing agents used in drug delivery through the skin. The penetration enhancing agent could be a sulphoxide compound, such as dimethyl sulfoxide (DMSO). The penetration enhancing agent could be an azone compound, such as 1-dodecylazacycloheptan-2-one or laurocapram. The penetration enhancing agent could be a pyrrolidone compound, such as N-methyl-2-pyrolidone or 2-pyrrolidone. The penetration enhancing agent could be a fatty acid compound, such as lauric acid, oleic acid, myristic acid, or capric acid. The penetration enhancing agent could be a glycol compound, such as diethylene glycol or tetraethylene glycol. The penetration enhancing agent could be a surfactant compound, such as polyoxyethylene-2-oleyl ether or polyoxyethylene-2-stearyl ether. The penetration enhancing agent could be an essential oil, terpenes, or terpenoid compound such as oils extracted from eucalyptus, Chenopodium, and ylang-ylang.

Topical Carrier Vehicle. The ingredients are formulated into a topical composition by mixing with one or more carrier materials. The composition could be formulated into any suitable topical form that can be applied to the skin for therapeutic purposes. The formulation could be liquid, semi-liquid, or semi-solid form. Common types of topical formulations include ointments, gels, creams, lotions, foams, pastes, skin patches, sprays, etc. The ingredients are mixed with the carrier material (e.g. blended, dispersed therein, encapsulated therein, etc.).

The active agent can be admixed in the carrier material in any suitable way, including homogeneous admixtures, heterogeneous admixtures, or a combination thereof. The carrier materials could serve any suitable role such as an emulsifier, preservative, emollient, moisturizer, solubilizing agent that makes ingredients more soluble, solvent, humectant, thickener, gelling agent, etc.

Examples of carrier materials include water; alcohol solvents such as ethanol, isopropyl alcohol, benzyl alcohol; sodium hydroxide; potassium hydroxide; lanolin; polysorbates; propylene glycol; glycerin; triethanolamine; acrylic acid polymers such as carbomers; fatty acids, esters thereof, or lipid substances such as caprylic acid, stearic acid, isopropyl myristate, ethylhexyl palmitate, C₁₂₋₁₅ alkylbenzoate, glyceryl stearate, cocoa butter, beeswax, mineral oil; polyethylene glycol and derivatives thereof such as PEG-100, polyethylene glycol ester of stearic acid, PEG-10, polyethylene glycol derivatives of dimethicone; soybean sterols; isopropryl isostearate; petrolatum; cetyl alcohol; hyaluronic acids or salts thereof; ceramides; cholesterol; xanthan gum; pectin; and carboxymethyl cellulose.

Alternate Categories. The above-listed categorization of ingredients is not necessarily exclusive. For example, menthol could be considered as a topical analgesic or a penetration enhancer. In another example, aloe vera could be considered a botanical ingredient or topical analgesic.

Treatment Method. In another aspect, this invention is a method of treating a musculoskeletal problem (e.g. in a joint or nerve) in a patient using the topical therapeutic composition described herein. Any of various types of musculoskeletal symptoms could be treated such as pain, soreness, inflammation, swelling, redness, stiffness, etc. Any of various musculoskeletal tissue areas could be targeted for treatment, including muscles, tendons, cartilage, joints, nerves, etc. Examples of joints that could be targeted for treatment include knee, finger, wrist, lumbosacral spine, cervical spine, ankle, elbow, etc. Examples of nerves or nervous system problems that could be targeted for treatment including sciatica, peripheral neuritis or neuralgia, migraine, common headache, etc.

The method could comprise applying 0.25-3.0 mls or 0.4-4.5 grams of the composition to a joint area. The composition could be applied to the affected area one to four times daily. For convenience use, the topical composition could be provided in a topical medication dispenser (such as a dispensing tube). Expressed as weight, the medication dispenser could contain 30-200 grams of the topical composition. For example, the product could be a dispensing tube containing about 60 grams or about 100 grams of the topical composition. Expressed as volume, the medication dispenser could contain 15-250 mls of the topical composition. For example, the product could be a metered-dose dispenser loaded with 30-90 mls of the topical composition.

### DETAILED DESCRIPTION

The following are more detailed descriptions of the possible ingredients in the topical formulation of this invention. Non-Steroidal Anti-Inflammatory Drugs (NSAID). In this invention, the primary therapeutically active ingredient is an NSAID. This is a class of drugs which reduces pain, inflammation, and fever. The term “non-steroidal” is meant to distinguish from corticosteroids, which are also anti-inflammatory but have more prominent side effects. NSAIDs work by inhibiting cyclooxygenase enzymes (COX-1 and COX-2 enzymes). These enzymes are involved in the synthesis of prostaglandins. The inhibition of prostaglandin synthesis ultimately down-regulates biological mediators of pain and inflammation at the local level.

NSAIDs are used in a wide range of clinical setting, such as in the treatment of soft tissue pain and inflammation (e.g. tendonitis, soft tissue injury such as ankle sprains, low back pain, osteoarthritis, rheumatoid arthritis, headache, acute gout, menstrual pain, postoperative pain, etc). Some examples of NSAIDs are naproxen, ibuprofen, diclofenac, ketorolac, etodolac, oxaprozin, diflunisal, meloxicam, or piroxicam. Although highly effective, NSAIDs also have potentially significant adverse effects. The most serious adverse effects are gastrointestinal (e.g. gastric ulcers and bleeding), cardiac (e.g. heart attacks), and renal (e.g. reduced blood flow to kidneys, sodium retention).

Diclofenac is an NSAID that is sometimes used in treating osteoarthritis and pain management. Diclofenac is currently available in topical form for the treatment of osteoarthritis pain of the knees and hand joints. Topically applied diclofenac is proposed to exert local direct effect on the underlying joint. However, at least some of the effect of topical diclofenac is likely also due to its systemic absorption.

Diclofenac has serious disadvantages as an anti-inflammatory agent. One of the most concerning is the risk of serious cardiac events. Diclofenac promotes thrombosis, and this can increase the risk of heart attack. Although the risk may be small, widespread use may represent a large number of such serious cardiac events in absolute terms.

Naproxen. In this invention, the preferred NSAID is naproxen. This is because it is relatively safe and effective; and also has the benefit of a prolonged duration of action compared to other NSAIDs. Naproxen is particularly suitable for topical formulation because it is also capable of penetrating through the stratum corneum of the skin, and thereby have a local (as well as systemic) effect against pain and inflammation. Research indicates that naproxen is further absorbed systemically after topical administration and that topical naproxen is better absorbed than diclofenac and indomethacin. The naproxen could be in any suitable form including free acid or sodium salt. Naproxen sodium is preferred because it has higher high permeability across human skin.

Applying naproxen topically also has the benefit of reducing gastrointestinal, renal, hepatic, and cardiac side effects that are common to all NSAIDs. Because of the high incidence of adverse effects of oral NSAIDs, the American College of Rheumatology recommends a trial of topical NSAID therapy for the treatments of knee osteoarthritis prior to the use of oral NSAIDs. Cardiovascular risk must be considered with any NSAID. However, naproxen is associated with the lowest overall cardiovascular risk compared to other NSAIDs. In fact, naproxen is associated with a reduced number of myocardial infarctions.

One reason that has discouraged development of a topical naproxen formulation is that the oral dose amount for naproxen is about 10 times greater compared to diclofenac. Thus, the assumption has been that a comparable 10-fold dose amount would be needed for therapeutic efficacy of topical naproxen. This is considered an overload for the topical formulation and would also require too much naproxen to be absorbed through the skin. However, this invention overcomes this obstacle by enhancing the skin absorption of naproxen by using it in combination with a penetration enhancing agent (in particular, menthol). See detailed explanation below.

Menthol. In this invention, menthol is considered a topical analgesic, or a penetration enhancing agent, or both. Menthol is a waxy organic compound (more specifically a monoterpenoid) made synthetically or obtained from the oils of various mint plants, such as peppermint. Menthol is a crystalline solid at room temperature, but melts slightly above that. For medical use, menthol can serve as a topical analgesic. Menthol's analgesic effect is mediated through various mechanisms of action. Menthol can chemically trigger the cold-sensitive TRPM8 receptors in the skin (also known as cold & menthol receptor 1 because it is a skin thermoreceptor for cold that is also activated by menthol). This action is responsible for the well-known cooling sensation that menthol provokes when applied to the skin.

Menthol's other analgesic effects are mediated by selective activation of K-opioid receptors. Menthol also blocks calcium channels and voltage-sensitive sodium channels, thereby reducing neural activity that may otherwise stimulate muscle contraction. This results in a muscle relaxing effect. Additionally, menthol produces a central analgesic effect by activating GABA_{A} receptors, and blocking volage-gated sodium and calcium channels in the dorsal horn of the spinal cord.

Menthol can also act as a skin penetration enhancer for topically applied medications. The stratum corneum layer of the skin, which contains a mixture of hydrophobic lipids and keratin, acts as a penetration barrier. This restricts the flow of most polar compounds from penetrating through the epidermal layer of the skin. For many topical medications, menthol increases the flux across the stratum corneum, thereby enhancing skin penetration of the medication. This is especially effective when used with a polar solvent (such as ethanol, propylene glycol, or water). Although menthol is hydrophobic, this effect probably occurs through hydrogen bonding of the hydroxyl group of menthol with the polar solvent to form an amphiphile.

Camphor. In this invention, camphor is considered a topical analgesic. Camphor is a waxy, colorless solid that is classified as a terpenoid and a cyclic ketone. It is found in various plant sources, but can also be synthetically produced. Camphor has a long history of use as a topical medication and is commonly used as a skin cream or ointment to relieve itching from insect bites, minor skin irritation, or joint pain. It is absorbed in the skin epidermis, where it stimulates nerve endings sensitive to heat and cold. This produces a warming or cooling sensation when applied to skin. Its action on nerve endings also induces local analgesia.

Salicylates. In this invention, salicylates are considered topical analgesics. Examples of salicylates include acetylsalicylic acid (aspirin), sodium salicylate, salsalate, and sulfasalazine. The preferred salicylate in this invention is methyl salicylate. Methyl salicylate has long been used as a medication. Today, it is commonly used as a topical analgesic (such as the BENGAY® brand skin cream) in concentrations as high as 30%. Methyl salicylate easily penetrates the skin. A considerable amount of salicylate may be absorbed through the skin after topical application of methyl salicylate products. In fact, so much that acute salicylate poisoning has occurred following topical application of methyl salicylate.

Dimethyl Sulfoxide (DMSO). In this invention, DMSO is considered a skin penetration enhancing agent. DMSO is a small amphiphilic organosulfur compound that forms a colorless liquid. DMSO's amphiphilic property gives it the ability to function as a skin penetration enhancer for both hydrophilic and hydrophobic compounds that would not normally penetrate the stratum corneum of the skin. Thus, as DMSO penetrates through the skin, it is capable of carrying other therapeutic agents therethrough as well. This effect may be enhanced with the addition of EDTA to the topical formulation.

In this invention, using DMSO in combination with menthol as dual penetration enhancing agents may be particularly beneficial. By having menthol as a separate penetration enhancing agent, the concentration of DMSO in the topical formulation could be reduced. For example, whereas typical topical formulations use DMSO at about 46%, the menthol combination could reduce the DMSO needed to 1-20% range. Using a lower concentration of DMSO has the benefit of less irritation to the skin and less unpleasant sulfide odor. In addition, using DMSO in combination with menthol diminishes the strong odor of both menthol and DMSO because the strong intermolecular forces between the two compounds lowers the vapor pressure of both, thereby reducing their diffusion into the ambient air.

Arnica Montana. In this invention, Arnica Montana is considered a therapeutically active ingredient of botanical origin. Arnica Montana is also known as wolf's bane or leopard's bane and is a flowering plant in the daisy family Asteraceae. Arnica Montana can be used as an herbal medicine for analgesic and anti-inflammatory purposes. Numerous potentially therapeutically active substances are present in Arnica Montana. The main potentially therapeutic constituents of Arnica Montana are essential oils, fatty acids, thymol, pseudoguaianolide sesquiterpene lactones, and flavanone glycosides. Pseudoguaianolide sesquiterpenes constitute 0.2-0.8% of the flower head. They are the toxin helenalin and its fatty esters. Although toxic, helenalin possesses some in vitro anti-inflammatory and anti-neoplastic effects. Helenalin can also inhibit 5-lipoxygenase and leukotriene C4 synthase.

In particular, sesquiterpene lactones may be of particular interest in this invention because their anti-inflammatory effect operates through a different mechanism than NSAIDs. These lactones easily penetrate the skin and significantly reduce NF-κB mediated inflammation. Helenalin blocks NF-κB driven gene expression in a precise and specific manner. Arnica Montana gel preparations are available for use in treating musculoskeletal pain. In particular, Arnica Montana gel is effective in treating osteoarthritis of the hand with improved functional capacity, reduced duration and extent of morning stiffness, reduced intensity of pain, and fewer painful joints. This study also demonstrated that Arnica Montana topical gel was not inferior to oral ibuprofen.

Burdock (Arctium Lappa) Root. In this invention, Burdock root is considered a therapeutically active ingredient of botanical origin. Like Arnica Montana, Burdock is a plant in the Asteraceae family. The root has use in traditional Chinese medicine as a diuretic and fever reducer. Burdock root contains multiple different antioxidants including quercetin, luteolin, and phenolic acids. Burdock roots also contain mucilage, various sulfurous acetylene compounds, polyacetylenes, and guaianolide-type constituents. Burdock root has anti-inflammatory effects and may benefit inflammatory skin conditions such as acne or eczema. Relevant to this invention, research shows that Burdock root may have therapeutic efficacy in the treatment of osteoarthritis.

Quercetin. In this invention, quercetin is considered an anti-inflammatory agent. Quercetin is a plant flavonoid that has several important physiological actions on skin. Among those most relevant to this invention are its various anti-inflammatory actions. It inhibits actions on NF-κB and the release of several proinflammatory cytokines. This anti-inflammatory property suggests that quercetin would be effective in the treatment of osteoarthritis. Quercetin may have the additional benefit of repairing cartilage and promoting remodeling of osteoarthritic joints. Quercetin may suppress cartilage matrix degradation, production of proinflammatory mediators, chondrocyte apoptosis (cartilage cells which produce and maintain the cartilaginous matrix, which consists mainly of collagen and proteoglycans), and activation of synovial macrophages. These are all processes involved in the pathogenesis of osteoarthritis, and suppression thereof promotes chondrocyte growth. However, quercetin has poor water solubility and is unable to penetrate skin. Thus, topical quercetin requires the presence of a penetration enhancing agent for effective transdermal delivery.

Cromolyn. In this invention, cromolyn is considered an inflammatory agent. Cromolyn is most commonly identified as a mast cell stabilizer. Mast cells are involved in initiating inflammation through several mechanisms, including release of vasoactive and inflammatory mediators such as histamine, prostaglandin D, and leukotrienes. Cromolyn blocks the release of such inflammatory mediators from mast cells, thereby preventing activation of H₁, 5-HT₂, and 5-HT₃ receptors. These receptors are present in primary afferent neurons and responsible for various types of pain sensations. Thus, preventing activation thereof reduces pain sensations.

When applied topically, cromolyn may be particularly effective when used in combination with a penetration enhancing agent for treating an inflamed joint. Having cromolyn penetrate through the skin can be effective in stabilizing mast cells in and around the joint and the nerves surrounding the joint, thereby decreasing inflammation and pain. It is possible that cromolyn could produce this effect through different complementary mechanisms than NSAIDs.

Alzheimer Disease. The topical therapeutic formulation could also be useful for treating or preventing Alzheimer disease (AD). Multiple epidemiologic studies indicate that long term use of NSAIDs drastically reduces AD risk in the elderly population, including delaying onset of disease, reducing symptomatic severity, and slowing cognitive decline. This risk reduction may occur by suppression of COX activity, which may prevent or reduce microglial activation and cytokine production in the brain or PGE2 responses in synapses. NSAIDs may also reduce amyloid production independent of COX activity. Asymptomatic high-risk individuals treated with conventional NSAIDs (such as naproxen) show reduced incidence of AD, but only after an interval of 2 to 3 years. CSF biomarker results support the hypothesis that naproxen can protect cognitively healthy persons against AD neurodegeneration. The biomarker data came from an independent sample of 117 dementia-free participants studied 21-41 months after the treatments had been terminated. Tau in CSF increases in patients with AD and in those with prodromal AD symptoms. In contrast, β-amyloid-(1-42) levels in CSF declines during the course of AD. A 2013 follow-up study did not favor treatment with NSAIDs, but the study discussion admits to multiple study issues that may have negatively skewed the results.

Experimental Work Prototype topical formulations were made to validate this invention. Note that in making the topical formulations below, the mixture often contracted in volume as the ingredients were blended together. This is likely from hydrogen bonding of the hydroxyl group (—OH) on the naproxen compound to the amine or hydroxyl groups contained in the added ingredients. This volume contraction is especially prominent with menthol and corroborates the ability of menthol to carry naproxen along as it penetrates through the skin. All concentration amounts are expressed as w/w %. Naproxen powder was obtained from tablets that were ground into a fine powder using a mortar & pestle.

Example #1. Mixed 3.0 g naproxen powder with 60 ml of BENGAY® topical analgesic cream (containing 4% camphor, 10% menthol, and 30% methyl salicylate). Then added 5.0 g of vitamin E facial cream and 10 g of moisturizer containing caprylic acid. Then added 5 ml of 65% isopropyl alcohol. Mixture blended until a uniform cream emulsion formed. The cream had a total volume of 30 ml (some volume loss due to contraction). Subject patient had moderately severe arthritis of the lower back with right sciatic nerve compression, and moderately severe pain in the L3-S1 area of the back. This pain radiated to the right sciatic region of the leg. Applied about 2.5 ml of this cream to patient's mid and lower back. Patient reported feeling a cooling sensation over the treated area within 5 min. Pain decreased from 7/10 to 2/10 (on a standard 1-10 pain scale) within 20 min. This pain relief penetrated through the skin. Duration of pain relief was 4-5 hours at 85% of the initial effect.

Example #2. Used a similar cream as Example #1 with the same amount of naproxen and topical analgesic cream. However, this formulation omitted the additional vitamin E cream and moisturizer cream. Subject patient had osteoarthritis in the index finger that had swollen to 1.5x normal diameter with entrapment of the flexor ligaments that restricted flexion by 50%. Applied about 0.5 ml of the cream to the affected finger. Swelling resolved within 30 min and flexion improved to about 75% of normal.

Example #3. Subject patient had moderate-severe lower back pain from osteoarthritis in the L2-S1 vertebrae. Patient had undergone L4-S1 laminectomy two years prior. About 0.75 ml of the cream of Example #2 was applied to the affected area in the lower back. Patient reported cooling sensation and mild pain relief within 30 min.

Example #4. Subject patient had moderate-severe pain in the right knee from osteoarthritis and a mild meniscus tear. Applied about 0.75 ml of the cream in Example #2 onto the affected knee. Patient reported a cooling sensation and mild relief of pain within 5 min. Patient further reported significant relief of deep pain (reduced from 8/10 to 3/10) and a mild reduction in knee joint swelling within 30 min. that was sustained for 3-4 hours duration.

Example #5. Mixed 3.0 g naproxen powder with about 45 ml of SOOTHING TOUCH® menthol cold freeze gel containing menthol, Arnica Montana, and Burdock (Arctium Lappa root) in filtered water, denatured alcohol, glycerin, triethanolamine, and carbomer 940. Mixture blended until a homogenous non-viscous cream resulted. Because of volume contraction from the mixture of ingredients, the resulting volume was about 30 ml. Subject patient had moderate lower back pain from osteoarthritis and nerve root compression. Applied a small amount of the cream to the lower back. Patient reported cooling sensation within 15 min. Patient further reported reduction of deep pain to 50-75% that was sustained for 3-4 hours duration. Repeated this treatment up to 5 times a day for about one month duration with continued mild pain relief. The patient (age 70) had laboratory testing at 1 and 6 months of treatment. The test results were a normal urinalysis, normal blood counts, normal kidney function parameters (BUN and creatinine), normal liver function parameters, normal lipid levels, and normal serum electrolytes. Also, the patient's blood pressure remained normal.

Example #6. Mixed 3.0 g naproxen powder with 50 ml BIOFREEZE® gel containing menthol 4% from aloe leaf extract, Arctium Lappa root (Burdock) extract, Arnica Montana flower extract, Boswellia Carterii resin extract, Calendula Officinalis extract, Camellia Sinensis (green tea) leaf extract, camphor, glycerin, Ilex Paraguariensis leaf extract, isopropyl alcohol, isopropyl myristate, Melissa Officinalis (Lemon Balm) leaf extract, silica, tocopheryl acetate, triethanolamine, and water. This yielded a 30 ml emulsion (note again, volume contraction). Made an additional 30 ml of the same formulation and stored in refrigerator at 4° C. Both the refrigerated gel and the ambient temperature gel remained stable for 30 days (as determined by continued clinical effectiveness). Applied this formulation to a subject patient, who reported immediate soothing and anesthetic cooling sensation, similar to Examples #1-5 above. Patient further reported that these effects were more prominent for the refrigerated sample.

Example #7. Mixed 3.0 g naproxen powder with 45 ml of SOOTHING TOUCH® menthol cold freeze gel containing menthol, Arnica Montana, and Burdock (Arctium Lappa) root, along with filtered water, denatured alcohol, glycerin, triethanolamine, and carbomer 940. This yielded a white emulsion with contracted volume of 30 ml. Poured the emulsion into a metered cream/gel dispenser. Subject patient had moderate-severe pain from osteoarthritis of the lower back with nerve root compression. Dispensed 0.75-1.0 ml of the emulsion to patient's lower back. Patient reported mild pain reduction associated with a cooling sensation after 5-15 min. Patient reported further reduction of pain by 75-90% after 30-45 min and was especially effective for deep aching pain. Duration of pain relief was about 4 hours. Patient used the formulation up to 4 times daily for 45 days duration. The only side effect was occasional erythema of the skin and reduced sensation to touch on the skin where the formulation was applied.

Example #8. Subject patient had osteoarthritis of the index finger with pain, joint swelling, reduced joint flexion by 75%. Applied the emulsion of Example #7 to the affected finger. Pain and swelling reduced by 75% and joint flexion improved by 50-75% after 30 min.

Example #9. To make a simple topical patch, spread 0.75 ml of the emulsion in Example #7 to adhesive bandages. Subject patient had a prior L4-S1 laminectomy for arthritis and disk rupture with subsequent pars fracture of the spine. This was associated with intermittent severe low back pain and sciatica that caused tingling in the feet. Applied the emulsion-coated bandages over the L3-S2 areas of the back. The back pain resolved (although tingling in the feet continued) after 5 min. Pain relief continued for 3 hours and 15 min. At 3.5 hours, the back pain returned as the coated bandages began to peel off. There was no skin reaction. Reapplied the emulsion to the adhesive bandages for repeat treatment. The back pain diminished by 75-95% again within 5 min.

Example #10. Mixed 3.0 g naproxen powder with about 10 ml of ICY HOT® foam (containing 16% menthol). Further mixed with 10 ml of CERAVE© daily moisturizer (containing 3 essential ceramides plus hyaluronic acid). Repeated the addition of ICY HOT® foam 10 ml and 10 ml of CERAVE® daily moisturizer two more times with mixing. Then added 2 ml of DMSO with further mixing. This yielded a 40 ml emulsion. Loaded 30 ml of this emulsion into a topical dispenser device that dispenses 0.25 ml per click twist. Subject patient had osteoarthritis in both knees and a meniscal tear of the right knee. Patient was barely able to walk because of knee pain. Applied about 1 ml to each knee. Patient was able to walk with about 70% pain reduction after 30 min.

Example #11. Subject patient had sciatica and osteoarthritis of the lower spine with back pain, and had three prior surgical procedures on his back. Applied about 1.5 ml of the emulsion from Example #10 above to the patient's lower back. Patient experienced 80% reduction of back pain after 30 min. Patient continued use for up to 4x/day for 4 weeks duration without any skin irritation.

Example #12. Subject patient had osteoarthritis in both hands with both index fingers swollen to 1.5x normal diameter and flexion reduced to 60% of normal. Applied 1 ml of emulsion from Example #10 above to both hands. Swelling reduced by 60% and flexion improved to 90% of normal within 20 min. The emulsion had no unpleasant odor of DMSO or strong menthol smell. This is an important patient usability factor for topical treatments on hands and fingers. (In contrast, a different test formulation containing 5% DMSO had detectable DMSO odor.)

Example #13. Mixed 3.0 g naproxen powder with about 5 ml of DMSO, plus 20 ml of generic brand oil-free hypoallergenic facial moisturizer (containing water, glycerin, ethylhexyl palmitate, dimethicone, petrolatum, cyclomethicone, glycine, soybean sterols, isopropyl isostearate, cetyl alcohol, PEG-10 soy sterol, glyceryl stearate, PEG-100 stearate, C12-15 alkyl benzoate, carbomer, tetrasodium EDTA, sodium hydroxide, benzyl alcohol, chlorphenesin), plus 15 ml of CERAVE® daily moisturizer (ingredients noted above in Example #10), plus 7.5 ml of generic brand cold & hot spray (containing menthol 7.5%, methyl salicylate 29.0%, denatured alcohol, Arnica Montana flower extract, Boswellia Serrata gum extract, dimethyl sulfone, Eucalyptus globules leaf oil, paraguariensis leaf extract, peppermint oil, olive fruit oil, propylene glycol, water). This yielded a white emulsion. Subject patient had osteoarthritis of the lower spine with moderately severe back pain. Applied 1.5 ml to the L2-S3 area. Patient reported a warm sensation at the site after 10 min. Patient reported an 80% reduction in back pain after another 10 min.

Example #14. Subject patient had osteoarthritis in both hands with stiffness and swelling in both index fingers. Applied the cream from Example #13 above to both hands of the patient (about 0.5 ml for each hand). Patient reported 95% improvement in stiffness of both index fingers after 30 min.

Example #15. Mixed 3.0 g naproxen powder with 15 ml of ARNICARE© gel (7% Arnica Montana, plus alcohol, carbomer, water, and NaOH). Then further mixed with 10 ml of ICY HOT® foam (16% menthol). Then further mixed again with addition of the same volume of ARNICARE® and ICY HOT® foam. This yielded a homogenous white cream with distinct menthol odor with volume contraction to 28 ml. Subject patient had osteoarthritis in both knees and a meniscal tear of the right knee. Applied 0.5 ml of the cream to patient's right knee. The patient reported a cooling sensation within a few minutes; and 50% pain reduction in the treated knee after 30 min.

Example #16. Subject patient had lower back pain and had spine surgery 8 months prior for spondylolisthesis, pars fracture, and bilateral sciatic nerve compression. Applied about 1 ml of the cream from above Example #15 to the lower back. Patient reported 40-50% pain reduction after 30 min.

Example #17. Mixed the same proportions and ingredients as in Example #15 above, except used a total of 40 ml of ARNICARE® gel and added 3 ml of DMSO. This yielded a lotion with a final volume of about 30 ml. There was no significant menthol or DMSO odor. This beneficial property results from strong hydrogen bonding with those ingredients, which decreases the vapor pressure thereof. Subject patient had osteoarthritis in both knees and a meniscal tear of the right knee. Applied the lotion to the right knee of the patient. Patient reported a cooling sensation and small reduction of pain after 5 min. Pain reduced by 70% after 30 min, which was sustained for 3-4 hours duration.

Example #18. Subject patient had lower back pain and had spine surgery 8 months prior for spondylolisthesis, pars fracture, and bilateral sciatic nerve compression. Applied about 1 ml of the lotion from Example #17 above to the lower back. Patient reported pain reduction by 75% after 30 min and was sustained for 3-4 hours duration. Patient continued to use the lotion up to 4 times/day for 5 days duration without any adverse effects.

Example #19. Mixed 3.0 g naproxen powder with 5 tsp of Arnica gel (containing 98% Arnica Montana extract) to yield white cream. Further mixed with 15 ml of ICY HOT® foam (16% menthol) to yield a uniform white cream of 30 ml total volume. Transferred cream to a topical dispenser device. Subject patient was a 68 year old woman with severe knee osteoarthritis requiring two arthroscopies for cleanout of debris and several knee injections. Applied 1.5 ml to both knees. Patient reported cooling sensation after 5 min. Further had 80% reduction in pain after 20 min that was sustained for 3-4 hours duration. Patient continued daily use for about 6 weeks until the entire 30 ml batch was completely used.

Example #20. Made another batch of the cream in Example #19 above. Subject patient was a 71 year old man with osteoarthritis of the index fingers in both hands, which limited flexion and caused occasional joint swelling. Applied 2.5 ml of the cream to each hand. Noted that swelling of both index fingers reduced by 80% after 10 min. Also, flexion improved by 100% on right index finger and by 85% on left index finger.

Example #21. Mixed about 3.4 g naproxen powder with 15 ml of Arnica gel (containing 98% Arnica Montana extract) and generic brand oil-free hypoallergenic moisturizer cream. Further mixed with 7 ml of ICY HOT® foam (16% menthol) to yield white cream. Further mixed with another 15 ml of ICY HOT® foam (16% menthol) to yield a uniform, slightly viscous white cream with slight pleasant odor of menthol and consistency of a conventional facial cream. Subject patient was a 71 year old man with chronic back pain with prior spinal fusion of L4-S1. Patient reported cooling sensation after 5 min. Further reported pain reduced by 60% after 15 min.

Example #22. Subject patient was the same 68 year old woman identified in Example #19. Applied the same cream as Example #19 to each hand. Noted finger mobility improved by 75% after 10 min.

The foregoing description and examples merely illustrate the invention and are not intended to be limiting. Each of the disclosed aspects and embodiments of the invention may be considered individually or in combination with other aspects, embodiments, and variations of the invention. Also, unless otherwise specified, the steps of the methods of the invention are not limited to any particular order of performance. Persons skilled in the art may perceive modifications to these embodiments that incorporate the spirit and substance of the invention. Such modifications are within the scope of the invention.

Any use of the word “or” herein is intended to be inclusive and is equivalent to the expression “and/or,” unless the context clearly indicates otherwise. As such, for example, the expression “A or B” means A, or B, or both A and B. Similarly, for example, the expression “A, B, or C” means A, or B, or C, or any combination thereof.

1. A topical therapeutic composition comprising:
naproxen in an amount of 1-25 wt %;
menthol in an amount of 1-25 wt %;
methyl salicylate in an amount of 10-50 wt %;
Arnica Montana or Burdock root in an amount of 1-15 wt %;
one or more carrier materials.

2. The therapeutic composition of claim 1, wherein the naproxen is naproxen sodium.

3. The therapeutic composition of claim 1, further comprising cromolyn.

4. The therapeutic composition of claim 1, further comprising quercetin.

5. The therapeutic composition of claim 1, further comprising camphor in an amount of 2-15 wt %.

6. The therapeutic composition of claim 1, further comprising a skin penetration enhancing agent.

7. The therapeutic composition of claim 6, wherein the skin penetration enhancing agent is dimethyl sulfoxide (DMSO).

8. The therapeutic composition of claim 7, wherein the DMSO is in an amount of 1-20 wt %.

9. The therapeutic composition of claim 1, further comprising another topical analgesic.

10. The therapeutic composition of claim 9, wherein the topical analgesic is camphor.

11. A method of treating or preventing a musculoskeletal problem or Alzheimer disease in a patient, comprising:
having a topical therapeutic composition comprising:
naproxen in an amount of 1-25 wt %;
menthol in an amount of 1-25 wt %;
Arnica Montana or Burdock root in an amount of 1-15 wt %;
one or more carrier materials;
applying the therapeutic composition onto the patient's skin.

12. The method of claim 11, wherein the therapeutic composition is applied in an amount of 0.4-4.5 grams.

13. The method of claim 11, wherein the therapeutic composition is applied in an amount of 0.25-2.5 mIs.

14. The method of claim 11, wherein the therapeutic composition is contained in a topical medication dispenser.

15. The method of claim 14, wherein the topical medication dispenser is a metered-dose dispenser.

16. The method of claim 11, wherein the naproxen is absorbed through the skin and into the patient's systemic circulation.

17. The method of claim 16, wherein the therapeutic composition is applied at a location of the musculoskeletal problem, and wherein the naproxen further has local effect at the location of the musculoskeletal problem.

18. The method of claim 17, wherein the location of the musculoskeletal problem is a joint.

19. The method of claim 18, wherein the musculoskeletal problem is osteoarthritis.

20. The method of claim 11, wherein the therapeutic composition further comprises methyl salicylate in an amount of 10-50 wt %.
